(19) United States
(12) Patent Application Publication
(10) Pub. No.: US 2006/0134066 A1
(43) Pub. Date: Jun. 22, 2006

(54) Localization of vectors and other agents

(76) Inventor: Gholam A. Peyman, New Orleans, LA (US)

Correspondence Address: WOOD, HERRON & EVANS, LLP, 2700 CAREW TOWER, 441 VINE STREET, CINCINNATI, OH, 45202, US

(21) Appl. No.: 11/021,198

(22) Filed: Dec. 22, 2004

(51) Int. Cl. A61K 9/127; A61K 9/14; A61K 48/00

(52) U.S. Cl. 424/93.2; 514/44; 424/489; 424/450; 977/916

(57) ABSTRACT

Methods are disclosed to enhance controlled localization and/or release of an agent at an anatomical and/or physiological site where the agent is desirable. In one embodiment, the method localizes vector transfected with a gene(s) that enhances neovascularization (i.e., genes encoding angiogenic agents). In another embodiment, the method localizes genes that inhibit neovascularization (i.e., genes encoding antiangiogenic agents) at sites where new blood vessel growth is undesirable. The vector is provided in a biocompatible substance substantially preventing migration of the vector from the site in need of therapy. The substance may be a matrix, gel, a polymer, liposome, capsule, nanoparticle, microparticle. The substance may form in situ, for example, a fibrin entraining mesh or network form from fibrinogen and thrombin.

### FIELD OF THE INVENTION

The invention is directed to localized delivery and placement of a physiological agent, such as localization in the eye of a vector containing a gene of interest.

### BACKGROUND

Gene delivery to specific ocular cells types and/or anatomic locations can reduce effects of ocular pathologies. Vectors such as viruses are used to contain genes that are being newly introduced into a cell or cell nucleus. The genes may themselves contain a modification that will achieve an effect. The genes may be unmodified but, once introduced into a cell or cell nucleus, they may achieve an effect.

The indiscriminate location of the vector, or release of the gene contained in the vector, can induce effects at locations where such effects are not needed. One example is gene therapy to provide angiogenic agents to facilitate vessel production in patients in need such therapy. For example, ocular pathology may cause the retinal artery to compress the retinal vein (central retinal vein occlusion, CRVO). In an attempt to compensate, the retinal vein drains through the optic nerve. However, blockage of the optic nerve reduces or prevents this blood flow, resulting in visual disturbances. In such patients, it is desirable to provide angiogenic gene therapy to treat or improve retinal vein flow. While useful in moderation, however, angiogenic gene therapy can cause abnormal blood vessels to bleed inside the eye. Thus, it is desirable to contain such agents at the desired site. Administration of gene vectors in the systemic circulation, or inside a cavity such as the eye, however, results in indiscriminate release of vectors in the body or inside body cavity.

Improvements are therefore desirable.

### SUMMARY OF THE INVENTION

One embodiment is a method to localize gene therapy. A vector transfected with the desired gene to achieve therapy is provided at a physiological or anatomical site where therapy is desired. As examples, a gene encoding an angiogenic factor is provided at a site where new blood vessels are desired (e.g., in the eye, in the heart, in the brain), or a gene encoding an antiangiogenic factor is provided at a site where it is desirable to inhibit blood vessels. In the inventive method, the vector is provided in or with a biocompatible substance that substantially prevents the transfected vector from leaving the specific site. The substance may be a matrix, gel, polymer, liposome, capsule, nanoparticle, and/or microparticle. In one embodiment, the substance is provided to the site in a “pro”-entraining form, and then forms the substance at the site where it is provided, for example, providing thrombin and fibrinogen, which then forms a fibrin entraining network in situ.

These and other advantages will be apparent in light of the following detailed description.

### DETAILED DESCRIPTION

Methods are disclosed to enhance controlled localization and/or release of an agent at an anatomical and/or physiological site where the agent is desirable. More specifically, the method enhances controlled localization, positioning or placement of, for example, a vector containing a gene at an anatomical and/or physiological site where it is desirable to locate the gene(s) provided by the vector. In one embodiment, the method localizes genes that enhance neovascularization (i.e., genes encoding angiogenic agents) by establishing new anastomoses within a system, or between two or more systems. In another embodiment, the method localizes genes that inhibit neovascularization (i.e., genes encoding antiangiogenic agents) at sites where new blood vessel growth is undesirable. The method enhances control and maintenance of vector localization, so that the vector does not significantly locate its associated gene(s) or gene product(s) (e.g., angiogenic agents or antiangiogenic agents) substantially beyond the desired specific site.

In one embodiment, a vector containing a gene encoding an angiogenic factor is controllably provided to a selective site where it is desirable to provide an angiogenic factor to establish new blood flow. In one example, blood flow is established between ocular nerves and the retinal circulation and/or the choroidal circulation in a patient with central branch or nerve occlusion. In this condition, blood flow is hampered by occlusion in the pathway of the returning nerve. Selectively locating gene(s) encoding angiogenic factor(s) can establish or create one or more new channels between the retinal nerve and choroidal circulation that previously did not exist.

In one embodiment, an incision is made in the choroid at the edge of the optic nerve sheath for placement. In another embodiment, placement is inside the retina in approximation to a major branch of the central retinal vein at the junction of the retina and choroid. Selective localization of a vector containing a gene encoding an angiogenic factor(s) enhances creation of choroid-retinal anastomoses. Thus, a controlled collateral route of blood flow is established to provide oxygen, nutrients, etc. from the blood where such a flow did not previously exist.

In one embodiment, the method is used to establish non-ocular neovascularization. As one example, the method can be used to localize vectors containing gene(s) encoding vasculogenic and/or angiogenic agent(s) at a site or sites where a body part (e.g., fingers, toes, ears, nose, limb, etc.) is to be reattached, or new blood vessel formation needs to be stimulated (e.g., patients with peripheral vascular disease) such that new vessels are desirable. As another example, the method can be used to localize these vectors at a transplant site (e.g., kidney, lung, skin, etc.) where new vessels are desirable. As another example, the method can be used to localize these vectors in the heart or brain to provide new vessels in patients with cardiac or cerebral ischemia, respectively.

In other embodiments, the method is used to localize vectors containing gene(s) encoding antiangiogenic agents (e.g., compounds that are capable of interacting with VEGFR-2 as receptor antagonists). Antiangiogenic agents would be useful in patients in which dysregulated angiogenesis is a characteristic pathology.

In other embodiments, the method is used to localize vectors containing gene(s) encoding neurotrophic factors to retinal pigment epithelial cells to inhibit the progress of retinitis pigmentosa.

The inventive method enhances agent containment by providing a material or substance by which or in which the vector is contained. The material or substances is any biocompatible material that will retain, entrain, encapsulate, contain, or localize the vector so that the vector is substantially localized at its site of initial administration. In one embodiment, the method provides controlled release of the contained agent.

Vectors that may be used include viral vectors. Non-limiting examples of viral vectors are known to one skilled in the art and include adenovirus, recombinant adenovirus, adeno-associated virus (AAV), lentiviruses, retrovirus, alphavirus, etc. Vectors that may be used also include non-viral gene delivery vectors. Non-limiting example of non-viral gene delivery vectors are known to one skilled in the art and include naked DNA, polycation condensed DNA linked or unlinked to killed adenovirus, small interfering RNAs (siRNAs, e.g., SeqWright Inc., Houston Tex.), etc.

In one embodiment, vectors that contain the gene of interest are provided with a substance that will not significantly spread or migrate after injection. They may be mixed into the substance, or may be provided essentially simultaneously with the substance. In one embodiment, the substance is one or more of a natural and/or synthetic semisolid, gel, hydrogel, colloid, reticular network, matrix, etc. In one embodiment, the substance forms in situ. In one embodiment, the substance is a hydrogel liquid below body temperature, but gels to form a shape-retaining semisolid hydrogel at or near body temperature. In one embodiment, the substance is polyethylene glycol (PEG). In one embodiment, the substance is one or more of polyanhydrides, polyorthoesters, polylactic acid and polyglycolic acid and copolymers thereof, collagen, protein polymers, polymers, copolymers, and derivatives of polyester, polyolefin, polyurethane, polystyrene, polyethylene glycol/polyethylene oxide, polyvinylalcohol, etc.

In one embodiment, the substance is a combination of fibrinogen and thrombin that, when mixed, forms a reticular or network structure (e.g., a fibrin network). As known to one skilled in the art, the structure of fibrin may be altered by varying the concentration of thrombin mixed with fibrinogen. Relatively lower thrombin concentrations produce relatively thicker fibrin fibrils with a larger pore size, slower setting rate, and slower degradation rate. Thus, the substance may be altered to contain a vector for a desired duration and with a desired durability, delivery rate, degradation rate, geometry, etc., as known to one skilled in the art. The vector(s) may be mixed with either fibrinogen and/or thrombin and injected together to create a vector entrapped inside the mesh of fibrin. Containment of the vectors at a desired site enhances control of the gene product, for example, by reduced spreading immediately after administration (e.g., injection, implantation, etc.).

The method may be used for delivering a vector containing any gene to inhibit or promote a process only at a defined physiological or anatomical location, e.g., at or in a defined area or tissue. The method may also be used for modifying release over time to provide sustained or controlled release. An extended release formulation is also termed a controlled release formulation, formulated so that the release of the agent occurs in an extended or controlled fashion to prevent a bolus introduction. An alternative embodiment is a delayed release formulation, formulated to minimize or prevent the agent at a site other than a desired site. Both extended release forms and delayed release forms are termed modified release forms.

In one embodiment, vectors are entrained in a microencapsulated form. Examples include liposomes, microspheres, microcapsules, etc. In one embodiment, vectors are contained in particles produced through nanotechnology. Examples include soft absorbant nanoparticles, and nanoparticles with rigid shells. Other examples may be a polyvinyl alcohol hydrogel with a diameter in the range of about 500 nm to about 750 nm; a poly-N-isopropylacrylamide N-isopropylacrylamide hydrogel (50 nm to 1 μm); a copolymer of poly(ethylene oxide)-poly(L-lactic acid); or poly(L-lactic acid) coated with poly(ethylene oxide). In another embodiment, the entrainment substance is a reservoir or depot for the vectors within an anatomical or physiological site.

In one embodiment, the substance itself is localized by or located in a containment substance. For non-limiting descriptive purposes, this embodiment may be considered as a “capsule within a capsule” or an “encapsulated capsule”. In this embodiment, the outer “capsule” is positioned at a desired anatomical or physiological location, e.g., by surgical placement, by injection, by topical suturing, etc. Once positioned, the inner “capsule” provides therapy (gene therapy, drug therapy, etc.). Upon need for additional or adjuvant therapy, the outer “capsule” is accessed at its desired site such that the inner “capsule” may be refilled, replaced, etc. without the need to disturb, relocate, remove, etc. the outer “capsule” from its desired location. The “capsule” may be positioned within a site to facilitate ease of additional “loading” of the active.

The invention will be further appreciated with reference to the following non-limiting examples.

### EXAMPLE 1

A recombinant adenovirus is used at a titer of 3.0×10¹⁰ plaque forming units (PFU)/ml. Two μl of a solution containing 6.0×10⁷ PFU/ml virus is delivered into the eye (e.g., subretinal space) using methods known to one skilled in the art. Successful administration is confirmed by the appearance of a subretinal bleb and/or partial retinal detachment, using an operating microscope or by indirect ophthalmoscopy.

Expression of a vector-contained reporter gene (e.g., green fluorescent protein, etc.) or gene of interest is observed by methods known to one skilled in the art, e.g., fundus fluorescent photography, other photography, Western blots of representative tissue samples, etc.).

### EXAMPLE 2

An injectable solution is prepared containing a viral vector (e.g., adenovirus vector) encoding a gene of interest (e.g., platelet derived growth factor (PDGF)) and fibrinogen (e.g., 100 μl 1-100 IU fibrinogen, or 4 IU, or 16 IU, or 4-20 IU). The gene of interest may be or may regulate vascular endothelial growth factor (VEGF), a central regulator of angiogenesis (formation of new blood vessels from pre-existing vessels), platelet derived growth factor (PDGF), and vasculogenesis (development of embryonic vasculature through an influence on endothelial cell differentiation and organization). The solution is injected into the eye at the site of interest (e.g., at the edge of the optic nerve sheath). A solution of thrombin is then injected at the same location at a concentration sufficient to activate conversion of fibrinogen to fibrin; such concentrations may be determined empirically as known to one skilled in the art. The resulting fibrin reticular structure entrains the vector at the ocular site requiring therapy and reduces the presence of the vector at other sites.

The following references are expressly incorporated by reference herein in their entirety: U.S. Patent Application Publication Nos. 2004/0254419; 2003/0045865; 2002/0151513; 2002/0194630; 2003/0087850; U.S. Pat. Nos. 6,818,753; 6,828,432; 6,824,791; 6,832,735; 5,593,974 and 5,827,702; Shen et al. Arch. Ophthalmology (2001)119,1033.

Other variations or embodiments of the invention will also be apparent to one of ordinary skill in the art from the above figures and descriptions. Thus, the forgoing embodiments are not to be construed as limiting the scope of this invention.

What is claimed is:

1. A method of localizing gene therapy comprising providing at a body site in need of therapy a vector transfected with a gene encoding a therapeutic agent, the vector provided In a biocompatible substance substantially localizing the vector at the site in need of therapy.

2. The method of claim 1 wherein the substance is at least one of a biocompatible matrix, a gel, a polymer, a liposome, a capsule, a nanoparticle, and/or a microparticle.

3. The method of claim 1 wherein the substance forms in situ.

4. The method of claim 1 wherein the vector is at least one of a virus or a plasmid.

5. The method of claim 1 wherein the gene encodes an agent selected from the group consisting of an agent that enhances angiogenesis and an agent that enhances antiangiogenesis.

6. The method of claim 1 wherein the gene encodes at least one of a vascular endothelial growth factor or a platelet derived growth factor.

7. A method of localizing gene therapy comprising administering at an ocular site in need of therapy a composition comprising a gene encoding a desired agent, fibrinogen, and thrombin in concentrations sufficient to convert fibrinogen to form a fibrin network, the fibrin network entraining the gene at the site in need of therapy.

8. (canceled)

9. The method of claim 7 wherein the gene encodes at least one of an angiogenic agent or an antiangiogenic agent.

10. The method of claim 7 wherein the gene encodes at least one of a vascular endothelial growth factor or a platelet derived growth factor.
